(19) United States
(12) Patent Application Publication
(10) Pub. No.: US 2024/0180570 A1
(43) Pub. Date: Jun. 6, 2024

(54) IMPLANT RETRIEVAL FORCEPS

(71) Applicant: Dan Rosen, Sherman Oaks, CA (US)

(72) Inventor: Dan Rosen, Sherman Oaks, CA (US)

(21) Appl. No.: 18/076,368

(22) Filed: Dec. 6, 2022

(51) Int. Cl. A61B 17/22; A61B 17/28

(52) U.S. Cl. CPC A61B 17/22031; A61B 17/2833; A61B 2017/22035

(57) ABSTRACT

An implant retrieving forceps apparatus for retrieving from a patient an improperly placed implant having a driving socket has a first handle portion and an opposing second handle portion. A first jaw and an opposing second jaw are hingedly connected to the first handle portion and the second handle portion. A first extension extends from the first jaw, the first extension having an engaging portion configured to engage the driving socket. A second extension and a third extension, both extending from the second jaw in a parallel co-terminus configuration. The second extension and the third extension are configured to grip the implant with the engaging portion in the driving socket when the first handle portion and the second handle portion are moved proximally.

### FIELD OF THE INVENTION

### Field of the Invention

The present invention generally relates to dental surgery and dental implants. More specifically, the present invention relates to a dental implant retrieving forceps for removing a dental implant from a patient

### Background

Implants in dentistry are widely known. Implants typically consist of the implant itself, which is a threaded piece having a driving bore and a seat. After a patient's bone is prepared, usually by drilling out a preferred implant site, the implant is screwed into position in a manner like a conventional screw. Once the bone has solidified around the implant, a patient may return for a subsequent procedure, in which an abutment is seated on the implant, and a prosthetic tooth seated on the abutment.

In certain instances, an implant site may be adjacent a hollow area, such as a sinus (for example, the maxillary sinus). In instances like these, an implant may be driven through the patient's bone to become too close to the sinus. In such a case, the implant is in danger of traveling fully through the bone and into the sinus, requiring surgery to remove it. When this happens, it is inadvisable to use a driving tool to retrieve the implant, since merely connecting the driving tool to the implant may cause the implant to travel into the sinus. Additionally, any pulling force on the driving tool while connected to the implant will typically cause the driving tool to simply dislodge itself from the implant.

Dental forceps are also well known in the art, and may be used in an attempt to retrieve an implant. Dental forceps as presently known are ill-suited to this task. Even if they have fine, small size pincers, the forceps will typically destroy sensitive tissues as a user tries to manipulate them around the outside of the implant. Additionally, the outside of the implant is generally slippery when installed in the patient, and the pincers may slide off for this reason. Finally, there is a risk that the opposing pressure of the pincers on the implant may cause them to slip off, thereby driving the implant deeper, in some cases irretrievably into a patient's sinus cavity or other hollow area.

Hence, what is needed is a forceps for securing dental implants that provides an effective way of securing the rear of the implant and removing it from a patient without the limitations of existing techniques.

### SUMMARY

An implant retrieving forceps apparatus is disclosed for retrieving an implant from a patient. The implant may be improperly placed, and may be flush with the patient's anatomy, thereby being difficult to otherwise remove. The implant includes a driving socket proximal a rear portion of the implant, opposite a tip and threads of the implant. The apparatus includes a first handle portion and an opposing second handle portion, as well as a first jaw and an opposing second jaw. The first handle portion and the second handle portion are together separated from the first jaw and the second jaw by a hinge, and configured such that the first jaw and the second jaw move apart when the first handle portion and the second handle portion move apart.

A first extension is provided, extending from the first jaw. The first extension terminates in an engaging portion configured to engage the driving socket. A second extension and a third extension, both extend from the second jaw. The second extension and the third extension are configured to grip the implant with the engaging portion in the driving socket when the first handle portion and the second handle portion are moved proximally closer to each other. Preferably the engaging portion is smaller in circumference than the driving socket, so it can be easily inserted therein. The first handle and the second handle preferably each comprise a first type of gripping surface configured to reduce slippage when the forceps is in a user's hands. Additionally, a locking feature may be provided for locking the first handle portion to the second handle portion to keep the forceps in a closed configuration.

The forceps may also include a stop configured to prevent the first handle and the second handle from moving proximal to each other past a predetermined distance. The first jaw and the second jaw preferably each comprise a second type of gripping surface, which is configured to reduce slippage between the forceps and objects grasped by the forceps. The first extension is preferably larger than the engaging portion, and the engaging portion preferably extends from the first extension opposite the hinge. The second extension and the third extension are preferably co-terminus to grasp the implant more effectively. Additionally, the engaging portion, the second extension, and the third extension are preferably co-terminus with each other.

In another implementation, the implant retrieving forceps apparatus for retrieving from a patient an improperly placed implant having a driving socket may be characterized as having a first handle portion and an opposing second handle portion. A first jaw and an opposing second jaw are connected to the first handle portion and the second handle portion with a hinge. A first extension extends from the first jaw. The first extension has an engaging portion configured to engage the driving socket. A second extension and a third extension both extend from the second jaw in a parallel co-terminus configuration. The second extension and the third extension are configured to grip the implant with the engaging portion in the driving socket when the first handle portion and the second handle portion are moved proximally.

### BRIEF DESCRIPTION OF THE FIGURES

FIG. 1 illustrates a perspective view of a dental implant retrieving forceps according to one embodiment of the present invention.

FIG. 2 illustrates a first perspective view of a first jaw, second jaw, and hinge portion of the dental implant retrieving forceps.

FIG. 3 illustrates a second perspective view of the first jaw, second jaw, and hinge portion of the dental implant retrieving forceps.

FIG. 4 illustrates a perspective view of the first jaw, second jaw, and hinge portion of the dental implant retrieving forceps in preparation for securing a dental implant.

FIG. 5 illustrates the dental implant retrieving forceps in a first implant securing position.

FIG. 6 illustrates the dental implant retrieving forceps in a second implant securing position.

FIG. 7 illustrates a side view of the second jaw, including two extensions extending therefrom.

FIG. 8 illustrates a side view of the first jaw, including an engaging portion.

### DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

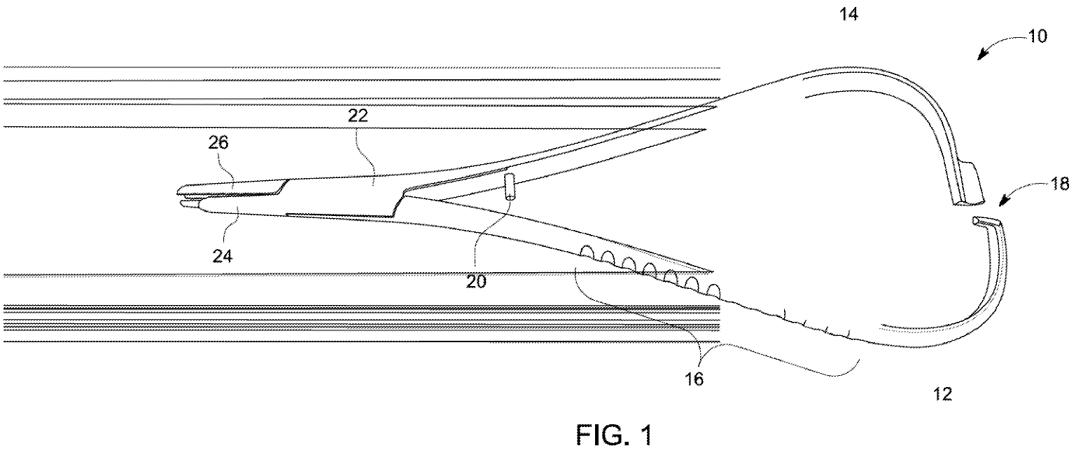

Referring to FIG. 1, an implant retrieving forceps 10 is shown the forceps 10 includes a first handle portion 12 and a second handle portion 14. The first handle portion 12 and the second handle portion 14 preferably each comprise a first gripping surface 16 to prevent the forceps 10 from slipping during use. The first handle portion 12 and the second handle portion 14 may also include a locking feature 18 enabling the first handle portion 12 and the second handle portion 14 to lock together, thereby preserving the forceps 10 in a gripping condition.

Still referring to FIG. 1, the forceps 10 may include a stop 20 to prevent over-flexing of the first handle portion 12 and the second handle portion 14. The stop 20 is proximal a hinge 22, from which a first jaw 24 and a second jaw 26 extend. In the illustrated embodiment, the hinge 22 is made of a nested configuration of the first handle portion 12 and the second handle portion 14, with the first handle portion 12 nested between areas of the second handle portion 14 to provide a smooth continuous surface when the forceps 10 is in a gripping configuration. The first jaw 24 and the second jaw 26 are preferably complimentary in size and come together with little overlap between them.

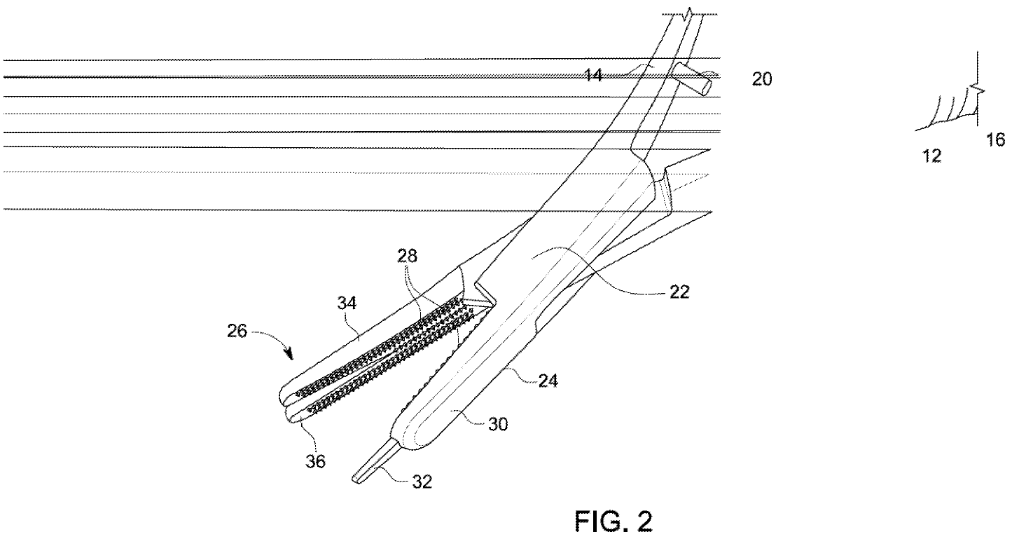

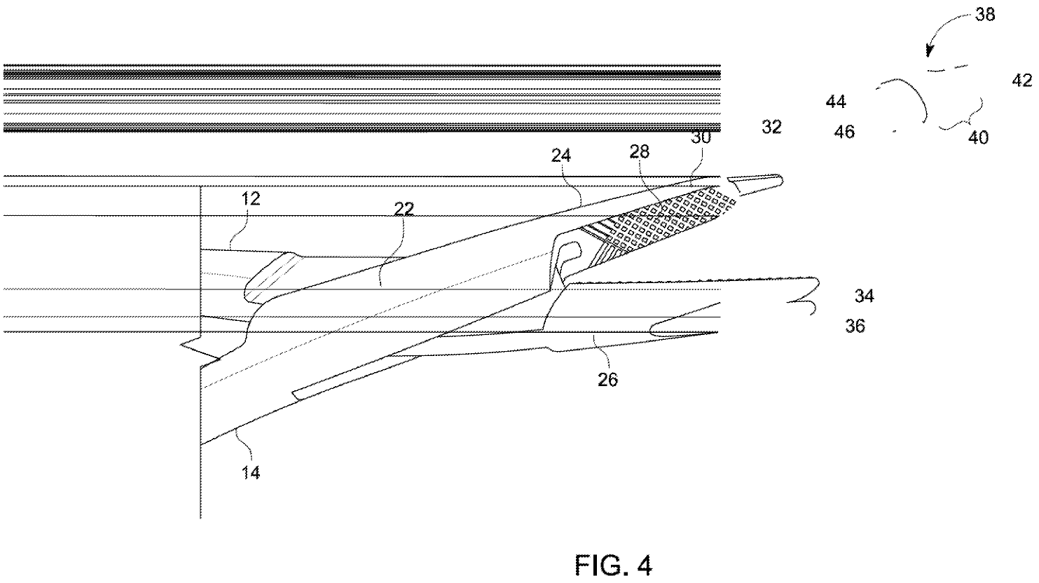

FIG. 2 illustrates a close-up enlarged view of the first jaw 24 and the second jaw 26. Both the first jaw 24 and the second jaw 26 each comprise a second gripping surface 28, which is more finely roughened than the first gripping surface 16, thereby enabling the forceps 10 to grip small articles and/or areas of the patient (other than a dental implant 38 FIGS. 4-6) under treatment more effectively. This enables the forceps 10 to be used as an implant retrieval system as well as conventionally for a variety of other purposes (e.g., gripping, manipulating, compressing, etc.)

Still referring to FIG. 2, the first jaw 24 has a single first extension 30 extending away from the hinge 22, and an engaging portion 32 extending from the first extension 30. The second jaw 26, in contrast, is bifurcated and comprises a second extension 34 and a third extension 36. Each of the first extension 30, second extension 34 and third extension 36 include the second gripping surfaces 28 discussed above. The second extension 34 and the third extension 36 preferably have the same length, and extend away from the hinge 22 an equal distance as the first extension 30 and engaging portion 32 together. Thus, when the first jaw 24 and second jaw 26 are closed together by bringing together the first handle portion 12 and the second handle portion 14, the engaging portion 32 of the first extension 30 is brought proximal to the terminal ends of the second extension 34 and third extension 36.

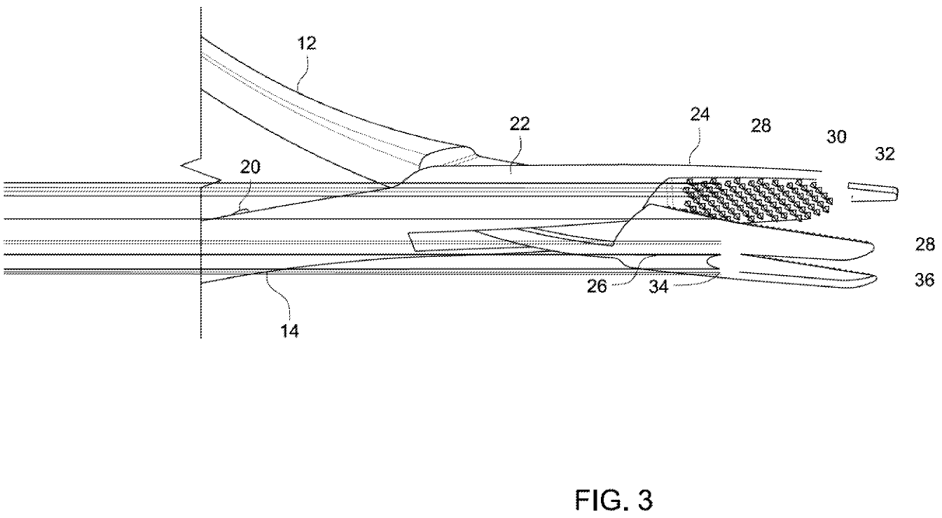

FIG. 3 illustrates the reverse of the close-up enlarged view of FIG. 2, to illustrate the second extension 34 and third extension 36 in more detail. The second extension 34 and third extension 36 are preferably spaced apart no farther than the width of the first extension, whereby the first jaw 24 and second jaw 26 have no overlap when closed. The spacing apart of the second extension 34 and the third extension 36 forms a slot between them. In the illustrated embodiment, the slot extends substantially far down the second jaw 26. In alternative embodiments, the slot may extend farther, such that the second jaw 26 essentially comprises only the second extension 34 and third extension 36. In other alternative embodiments, the second extension 34 and third extension 36 may be substantially shortened, such that the slot is shallow, without removing the slot entirely. Two points of contact must be provided (i.e., using the second extension 34 and the third extension 36) when contacting an implant 38 as discussed below.

FIG. 4 illustrates the reverse of the close-up enlarged view of FIG. 3 with a dental implant 38 ready for engagement and retrieval. The dental implant 38 includes a threaded portion having a tip 42 configured for engaging and anchorage in a patient's bone. A non-threaded rear portion 44 includes a driving socket 46 for driving the implant forward (or in reverse). The driving socket 46 may be configured in a variety of ways to have a driving profile. Typically, the driving socket is a conventional hex socket designed to receive a hex driver. Regardless of the driving profile of the driving socket 46, it is preferably larger than the engaging portion 32 of the first extension 30. This enables the engaging portion 32 to be easily inserted into the driving socket 46 and engage the implant 38.

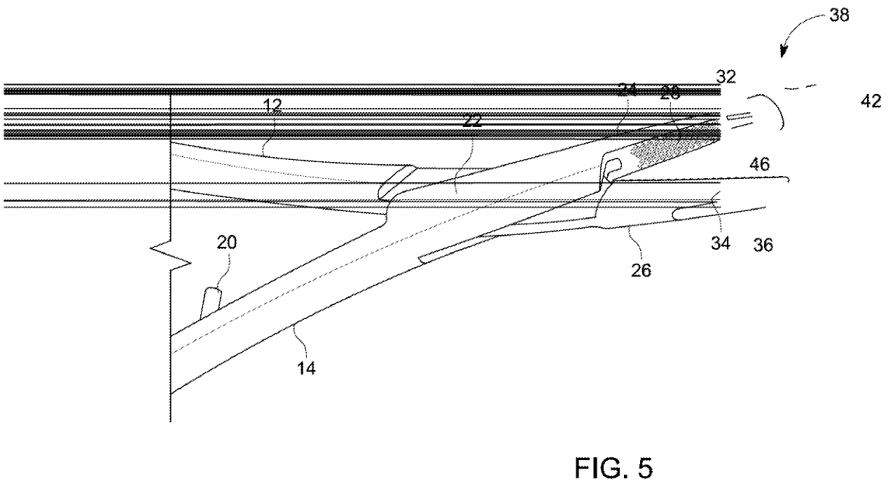

FIG. 5 illustrates the reverse of the close-up enlarged view of FIG. 3 with the dental implant 38 installed on the engaging portion 32. When the engaging portion of the first extension 30 is inserted into the driving socket 46 of the implant 38, the second extension 34 and the third extension 36 are in a position to close against the implant 38 and secure it. In the illustrated embodiment, this is because the second extension 34 and the third extension 36 are of co-equal length to the engaging portion 32. In alternative embodiments, the second extension 34 and the third extension 36 may extend farther than the engaging portion 32, or may not extend as far as the engaging portion 32. In either case they should be configured such that they grip the implant 38 when the engaging portion 32 is inserted therein.

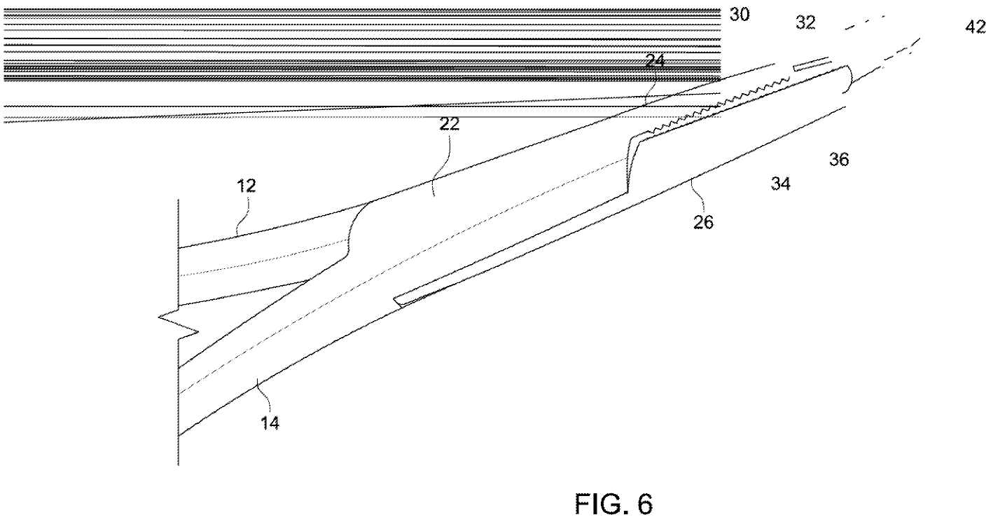

FIG. 6 illustrates the reverse of the close-up enlarged view of FIG. 3 with the dental implant 38 gripped by the forceps 10 and ready for retrieval. In this position the engaging portion 32 is installed in the driving socket 46 (FIGS. 4-5) and the second extension 34 and third extension 36 are each gripping the implant 38, thus holding it securely such that the forceps 10 can retrieve the implant 38 from a position, even if the implant 38 is confined within the patient. Although an implant 38 showing a rounded configuration and smooth rear portion 44 is shown, it is anticipated that any type of implant having a driving socket 46 may be secured by the forceps 10. By providing a second extension 34 and a third extension 36 on the second jaw 26, virtually any configuration of external profile may be grasped by the forceps 10.

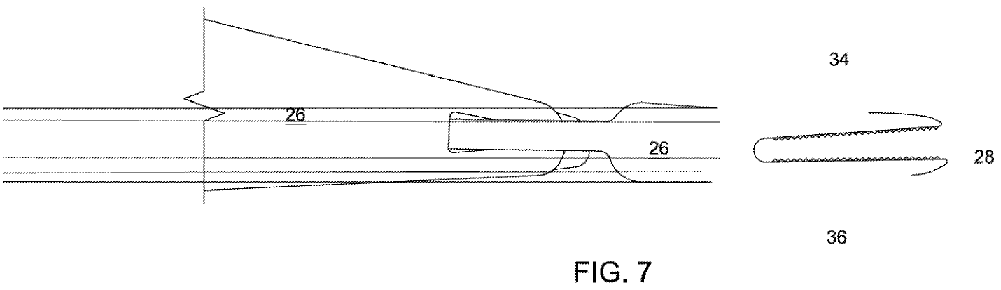

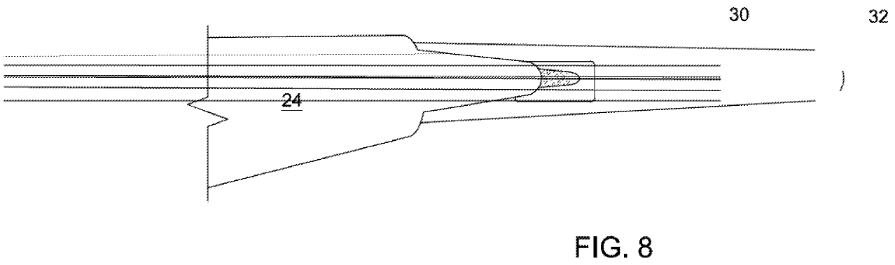

FIG. 7 illustrates a side view of the second jaw 26 including the second extension 34 and the third extension with a portion of the second gripping profile visible. FIG. 8 illustrates a side view of the first jaw 24, including the first extension 30, and the engaging portion 32 extending therefrom. Although an engaging portion 32 in the illustrated embodiment has a profile that reduces along its length, it may be configured in any way allowing the engaging portion 32 to be inserted into the driving socket 46 of an implant 48.

In order to employ the forceps 10, a user identifies an implant 38 that is improperly in position, either flush with, or impacted in a patient, including in soft and or thin areas of bone. Thereafter, the user opens the forceps 10 using the first handle portion 12 and the second handle portion 14, which may easily be accomplished singlehandedly. The engaging portion 32 is inserted into the driving socket 46 of the implant 38, and the second extension 34 and third extension 36 are aligned such that they will engage the outside of the implant 38. The first handle 12 and second handle 14 are then brought into a confined position. This has the effect of bringing the second extension 34 and the third extension 36 into contact with the implant 38 by virtue of the hinge 22. Optionally, the first handle portion 12 and second handle portion 14 may be locked together using the locking feature 18. The implant 38, now securely fastened in the forceps 10 may be pulled from its anchorage in the patient, and further remedial action taken.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. An implant retrieving forceps apparatus for retrieving from a patient an improperly placed implant having a driving socket, the apparatus comprising:
a first handle portion and an opposing second handle portion;
a first jaw and an opposing second jaw;
the first handle portion and the second handle portion, together separated from the first jaw and the second jaw by a hinge, and configured such that the first jaw and the second jaw move apart when the first handle portion and the second handle portion move apart;
a first extension extending from the first jaw, the first extension terminating in an engaging portion configured to engage the driving socket;
a second extension and a third extension, both extending from the second jaw; and
wherein the second extension and the third extension are configured to grip the implant with the engaging portion in the driving socket when the first handle portion and the second handle portion are moved proximally.

2. The apparatus of claim 1 wherein the engaging portion is smaller in circumference than the driving socket.

3. The apparatus of claim 1 wherein the first handle and the second handle each comprise a first gripping surface configured to reduce slippage.

4. The apparatus of claim 1 further comprising a locking feature for locking the first handle portion to the second handle portion.

5. The apparatus of claim 1 further comprising a stop configured to prevent the first handle and the second handle from moving proximal to each other past a predetermined distance.

6. The apparatus of claim 1 wherein the first jaw and the second jaw each comprise a second gripping surface configured to reduce slippage

7. The apparatus of claim 1 wherein the first extension is larger than the engaging portion, and the engaging portion extends from the first extension opposite the hinge.

8. The apparatus of claim 1 wherein the second extension and the third extension are co-terminus.

9. The apparatus of claim 1 wherein the engaging portion, the second extension, and the third extension are co-terminus with each other.

10. An implant retrieving forceps apparatus for retrieving from a patient an improperly placed implant having a driving socket, the apparatus comprising:
a first handle portion and an opposing second handle portion;
a first jaw and an opposing second jaw hingedly connected to the first handle portion and the second handle portion;
a first extension extending from the first jaw, the first extension having an engaging portion configured to engage the driving socket;
a second extension and a third extension, both extending from the second jaw in a parallel co-terminus configuration; and
wherein the second extension and the third extension are configured to grip the implant with the engaging portion in the driving socket when the first handle portion and the second handle portion are moved proximally.
